(19) United States
(12) Patent Application Publication
(10) Pub. No.: US 2019/0350610 A1
(43) Pub. Date: Nov. 21, 2019

(54) TISSUE HARVESTING DEVICES AND METHODS

(71) Applicant: The General Hospital Corporation, Boston, MA (US)

(72) Inventors: Alexander G. Sperry, Marlborough, MA (US); Charles R. Sperry, Springfield, VT (US); Dennis F. McNamara, JR., Walpole, NH (US); Brett Sitzlar, Uniontown, OH (US); Joshua Tam, Andover, MA (US); Richard Rox Anderson, Boston, MA (US)

(21) Appl. No.: 16/483,646

(22) PCT Filed: Jan. 23, 2018
(86) PCT No.: PCT/US18/14805

§ 371 Date: Aug. 5, 2019

Related U.S. Application Data
(60) Provisional application No. 62/455,002, filed on Feb. 6, 2017.
(60) Provisional application No. 62/479,761, filed on Mar. 31, 2017.

(51) Int. Cl. A61B 17/322; A61B 10/02

(52) U.S. Cl. CPC A61B 17/322; A61B 2010/0208; A61B 10/0266

(57) ABSTRACT

A tissue harvesting and wound closing device includes first and second actuator strip guides of a strip that are slideable along first and second panel guides of a panel, respectively. A first section of a base of the actuator strip has a substantially uniform width between the first and the second actuator strip guides. A second section of the base has a gradually decreasing width between the first and second actuator strip guides. A third second section of the base has a substantially uniform width between the first and second actuator strip guides that is smaller than the first width. When the actuator strip is pulled, the first and second actuator guides slide along the first and second panel guides, respectively, to cut a strip of tissue strip creating an open wound, and a panel pulls proximal tissue on opposite sides of the open wound to close the wound.

### CROSS-REFERENCE TO RELATED APPLICATIONS

This application is based upon and claims the benefit of U.S. provisional application number 62/455,002, filed on Feb. 6, 2017 and U.S. provisional application number 62/479,761, filed on Mar. 31, 2017, each of which is incorporated herein by reference and relied upon in its entirety.

### STATEMENT OF FEDERALLY SPONSORED RESEARCH OR DEVELOPMENT

Not applicable.

### BACKGROUND

The present disclosure generally relates to tissue harvesting devices, actuator strips, and methods for same.

Skin or tissue grafts involve the transplantation of tissue or skin. Tissue grafts can be used, for example, when a person has sustained a serious injury, undergone certain surgical procedures (e.g., wide local excision of a breast tumor or melanoma), has suffered radiation burn from cancer treatment, or has a chronic wound such as diabetic, pressure or venous ulcers resulting from neuropathy, vascular disease or other trauma. Before the skin or tissue can be transplanted in a graft procedure, the tissue must first be harvested from a donor site. Various devices exist for harvesting or cutting thin strips of tissue from donor sites, such as scalpels for full-thickness tissue grafts, and dermatomes for partial thickness grafts, but those devices can be difficult to use and do not provide an easy-to-use, single step sterile solution for cutting thin strips of tissue or for repairing damage done to the donor site tissue during the harvesting.

Improved tissue grafting methods, tissue arrangements for grafts, and tissue harvesting devices and methods for both full-thickness and partial thickness grafts are accordingly needed.

### SUMMARY

According to one embodiment of the present disclosure, a tissue harvesting and wound closing device includes a panel and an actuator strip. The panel includes first and second panel guides extending along at least a portion of a length of the panel. The actuator strip is coupled to the panel and includes a base. First and second actuator strip guides extend along at least a portion of the length of the base and are slideable along the first and second panel guides, respectively. A first section of the base has a substantially uniform width between the first and second actuator strip guides. A second section of the base has a gradually decreasing width between the first and second actuator strip guides. A third section of the base has a substantially uniform width between the first and second actuator strip guides that is smaller than the substantially uniform width of the first section. A cutter is coupled to the first section of the base. The device is configured such that when the device is secured to the donor site and the actuator strip is pulled, the first and second actuator guides slide along the first and second panel guides, respectively, such that the cutter cuts a strip of the tissue from the donor site creating an open wound, and the panel pulls together tissue proximal tissue on opposite sides of the open wound to at least partially close the open wound.

According to another embodiment of the present disclosure, a tissue harvesting and wound closure method is provided that includes securing a panel to a donor site and pulling first and second actuator strip guides along first and second panel guides of the panel, respectively, to (i) cut a strip of tissue from the donor site thereby creating an open wound and (ii) pull together proximal tissue on opposite sides of the open wound to at least partially close the open wound.

In yet another embodiment of the present disclosure, a tissue harvesting and wound closing actuator strip is provided that includes a base. The actuator strip further includes first and second actuator strip guides each extending along at least a portion of a length of the base and structured to be slideable along first and second panel guides of a panel, respectively. A first section of the base has a substantially uniform width between the first and second actuator strip guides. A second section of the base has a gradually decreasing width between the first and second actuator strip guides. A third section of the base has a substantially uniform width between the first and second actuator strip guides. A cutter is coupled to the first section of the base. When the actuator strip is secured to a donor site and the actuator strip is pulled, the first and second actuator guides slide along the first and second panel guides, respectively, such that the cutter cuts a strip of the tissue from the donor site thereby creating an open wound, and the panel pulls together proximal tissue on opposite sides of the open wound to at least partially close the open wound.

According to still another embodiment of the present disclosure, a tissue harvesting device includes a panel having first and second panel guides extending along at least a portion of a length of the panel. An actuator strip is coupled to the panel and includes a base. The actuator strip includes first and second actuator guides extending along at least a portion of a length of the base and slideable along the first and second panel guides, respectively. A bandage and an antiseptic and anesthetic/analgesic gel pack are positioned on the base. When the device is secured to the donor site and the actuator strip is pulled, the first and second actuator strip guides slide along the first and second panel guides, respectively, such that a cutter coupled to the base cuts the strip of tissue from the donor site thereby creating an open wound, the antiseptic and anesthetic/analgesic from the gel pack is applied to the donor site, and the bandage is placed over the open wound.

In yet another embodiment of the present disclosure, a tissue harvesting method includes securing a panel to a donor site, pulling first and second actuator strip guides of an actuator strip along first and second panel guides of the panel, respectively, to cut a strip of tissue from the donor site thereby creating an open wound, apply an antiseptic and anesthetic/analgesic to the donor site, and place a bandage over the open wound.

In still another embodiment, a skin grafting method includes coiling a full thickness strip of tissue, and placing the coiled full thickness tissue on a graft site.

### BRIEF DESCRIPTION OF THE DRAWINGS

FIG. 1 is a plan view illustrating one embodiment of a tissue harvesting and wound closing device of the present disclosure.

FIG. 2 is a perspective of an actuator strip of the tissue harvesting and wound closing device of FIG. 1.

FIG. 3 is perspective view of the tissue harvesting and wound closing device of FIG. 1 after the actuator strip has been pulled to cut a strip of tissue from a donor site and the actuator strip has been cut.

FIG. 4 is a cross-sectional view taken along B-B of FIG. 2 illustrating the actuator strip guides and panel guides of FIG. 1.

FIG. 5 is an enlarged plan view of a middle or second section of the actuator strip base of FIG. 1 showing a gradually decreasing width between the actuator guides.

FIG. 6 is an enlarged perspective view of a portion of the first section of the actuator strip of FIG. 2 showing the cutting device coupled thereto.

FIG. 7 is cross-sectional view taken along C-C of FIG. 6 illustrating the cutting device coupled to the actuator strip.

FIG. 8 illustrates the cutting device in a flat form, prior to being coupled to or being formed into the shape for being coupled to the actuator strip.

FIG. 9 is a perspective view of the cutting device prior to being coupled to the actuator strip.

FIG. 10 is a plan view of illustrating one embodiment of a tissue harvesting device of the present disclosure.

FIGS. 11 and 12 illustrate another cutting device of the present disclosure.

FIG. 13 illustrates an example of a harvested strip of full thickness tissue.

FIG. 14 illustrates a strip of full thickness tissue coiled into various arrangements according to embodiments of the present disclosure.

### DETAILED DESCRIPTION

This present disclosure provides tissue harvesting and wound closing devices, methods, and components for same that enable relatively thin strips of either partial thickness tissue or full-thickness tissue (e.g., skin containing epidermis and full dermis, and often some subcutaneous fat) to be easily harvested from a donor site. The harvested tissue can be arranged in a strip, a coil, a plug or can be stacked so that the arrangement can be used, for example, as a skin construct for grafting. The tissue harvesting and wound closing devices cut tissue from a donor site creating an open wound, which can be closed at substantially the same time that the tissue is being cut (i.e., in a single-step or substantially simultaneous process). In one aspect, a cutting device is integrated or coupled to an actuator strip that is secured to a donor site by, for example, an adhesive panel. Downward pressure can be applied to the device, if needed (e.g., exerting a force on the panel pieces to keep the device in place on the donor site), and guides extending along the length of the actuator strip can be pulled along cooperating guides or rails of the panel so that a thin strip of tissue or skin can be cut and harvested from a donor site in a controlled, precise manner. The pull or pulling that is performed in each of the devices, methods and actuator strips of the present disclosure is a generally continuous, smooth pulling action that is performed substantially along the longitudinal or lengthwise axis of the actuator. The devices and methods herein accordingly function such that a donor site can be opened and closed simultaneously and a thin strip of tissue can be harvested with minimal displacement of the surrounding donor site tissue. Once the cut has been made and the tissue has been harvested, the device can be left in place on the donor site so that the wound can heal with minimal scarring. The cutting device can be removed from the actuator strip after the cut has been made, by, for example, removing a portion of the actuator strip.

Alternatively or additionally, certain devices, actuator strips and methods herein allow a thin strip of tissue to be cut while an antiseptic and anesthetic/analgesic, as well as a bandage are applied substantially simultaneously with the cutting action so that the process of tissue harvesting can occur in a precisely controlled, single step, sterile process. If desired, scaffold/matrix materials can be combined with the harvested tissue strips to produce, for example, a skin construct having structural, nutritional, biochemical, or other forms of support. When the harvested tissue is full-thickness skin, the construct can greatly improve the quality of a post-graft skin, since the construct contains all components of the full-thickness skin, including dermal cells and adnexal structures that are usually missing from partial-thickness grafts or artificial skin substitutes.

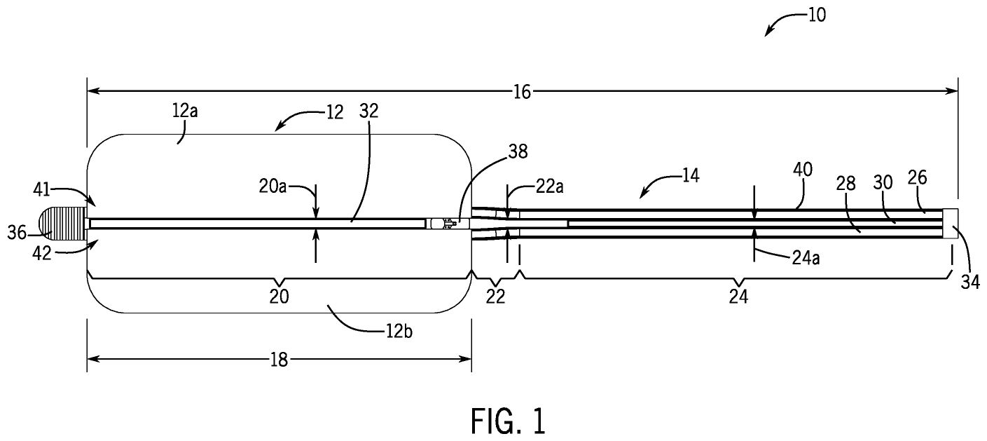

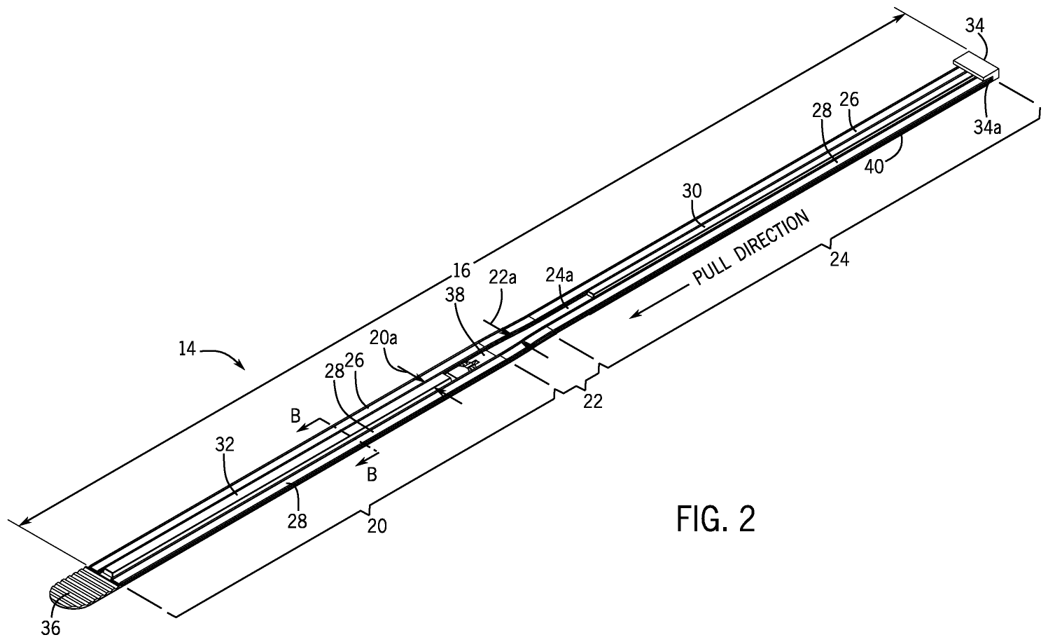

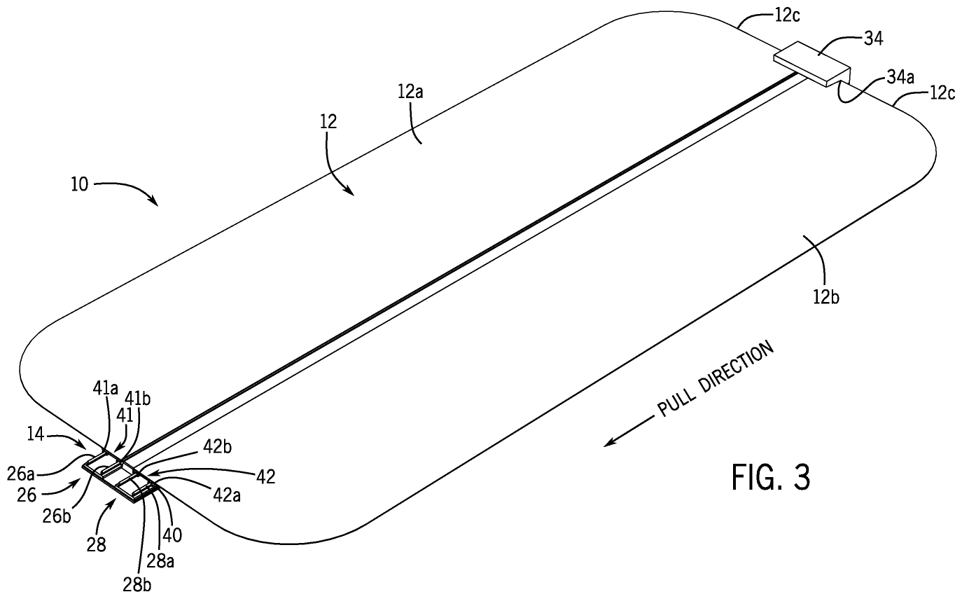

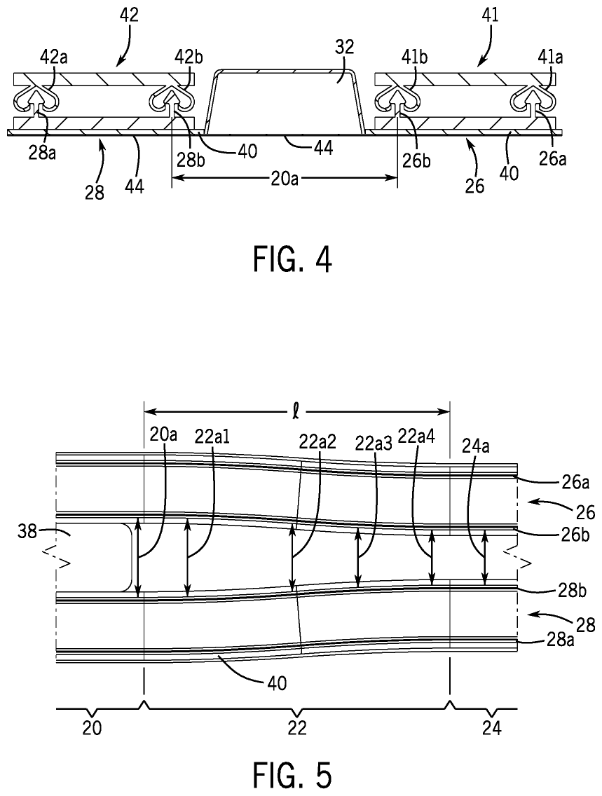

Turning now to the figures and referring primarily to FIGS. 1-4, one embodiment of a skin harvesting and wound closure device 10 of the present disclosure is illustrated. Device 10 generally includes a panel 12, and an actuator strip 14 coupled to or integrated with the panel. Panel 12 and actuator strip 14 are generally flat or straight, but can have any suitable geometric shape including curved, irregular or squiggly, to provide a more natural fit to the donor site and to reduce the visibility of the donor site scarring. Panel 12 has a length 18 and includes first and second panel pieces 12a, 12b, which can be separate pieces or a single integrated piece. An underside of panel 12 is structured to be secured to a tissue donor site. In one embodiment, the underside of panel 12 is generally flat and includes an adhesive for securing the panel 12 to the donor site from which tissue will be cut or harvested. Panel 12 can include a removable backing to protect the adhesive. Panel 12 further includes first and second panel guides 41, 42, which are illustrated at FIGS. 3 and 4 and discussed more below.

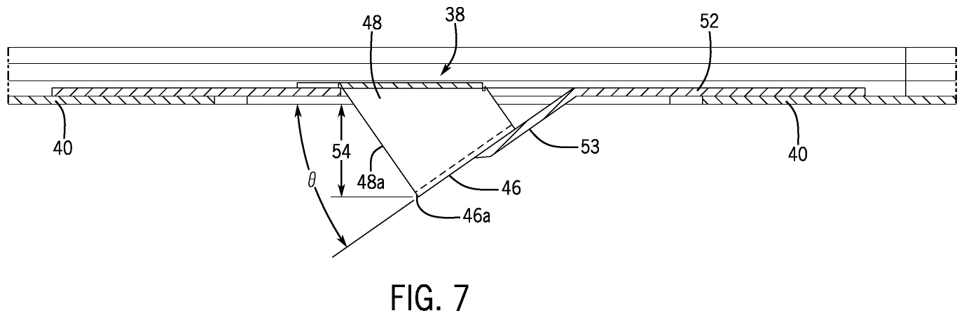

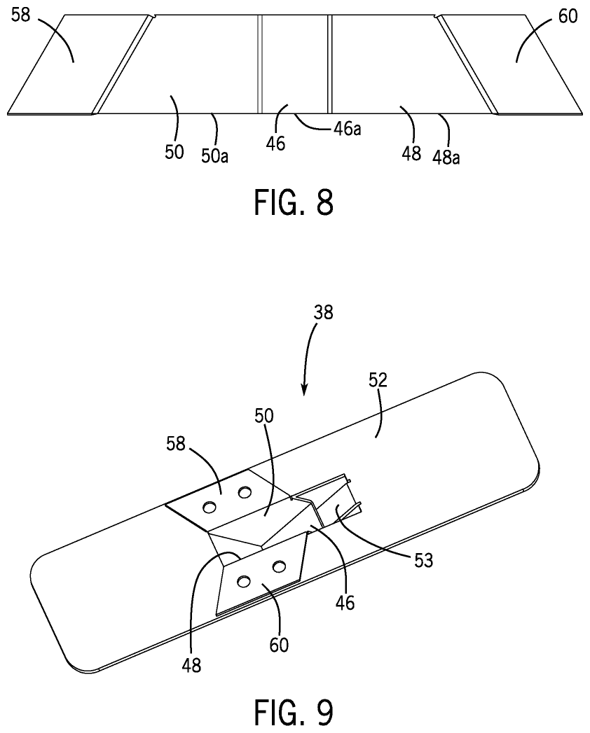

Actuator strip 14 includes a base 40 having a length 16 and defining first 20, second 22 and third sections 24. Length 16 can be any length suitable for harvesting or cutting a desired thin strip of tissue from a donor site, and in one embodiment can be approximately 450 mm (e.g., the first section 20 having a length of 200 mm, the second section 22 having a length of 25 mm, and the third section 24 having a length of 225 mm). First and second actuator strip guides 26, 28 each extend along the length of base 40, but in certain embodiments, do not extend along the entire length of the base 40, and instead extend only along a portion of the length. Actuator strip 14 further includes a stop 34 located at an end of the first section 20 of the actuator strip, and a pull tab 36 located at and end of the first section 20 of the actuator strip opposite the end at which the stop 34 is located. An anti-septic and anesthetic/analgesic gel pack 32 is located in the first section 20 of base 40, while a bandage 30 is located in the third section 24 of base 40. The anesthetic/analgesic in gel pack 32 can include an anesthetic alone, an analgesic alone or a combination of an anesthetic and an analgesic. Similarly, the anesthetic/analgesic used in any of the devices or methods described herein can include an anesthetic alone, an analgesic alone or a combination of an anesthetic and an analgesic. A cutter or cutting device 38 is coupled to the first section 20 of base 40 between the first and second actuator guide strips 26, 28, which can be located in a channel defined between the actuator guide strips 26, 28. Cutting device 38 is described in more detail below with respect to FIG. 7-9.

Regarding the first and second actuator strip guides 26, 28, and their cooperating panel guides 41, 42, first and second actuator strip guides 26, 28 are structured to be slideably coupled to or engaged with the first and second panel guides 41, 42. In the illustrated embodiment, first actuator guide 26 includes outer and inner actuator guides 26a, 26b, respectively, each of which extends along the length of base 40 and near or along an edge of base 40. The second actuator strip guide 28 likewise includes outer and inner actuator strip guides 28a, 28b, respectively, each of which extends along the length of base 40 and near or along an edge of base 40. First panel guide 41 similarly includes outer and inner panel guides 41a, 41b, respectively, each of which extends generally along the length of the panel 12 and outwardly from the underside portion of panel 12. Second panel guide 42 likewise includes outer and inner panel guides 42a, 42b, each of extends generally along the length of the panel 12 and generally outwardly from the underside portion of the panel 12. Actuator guides 26a, 26b, 28a, 28b can be in the form of a guide post or stick, while panel guides 42a, 42b can be in the form of clamping rails structured to interlock, clamp, or engage with the guide posts or sticks such that actuator guides 26a, 26b, 28a, 28b can guidingly move or slide along the respective panels guides 41a, 41b, 42a, 42b.

It should be appreciated that the first and second actuator strip guides and their respective cooperating panel guides can be any structure suitable to allow the actuator strip guides to guidingly engage and slide or move along the panel guides. In one embodiment, instead of the first actuator strip guide having inner and outer actuator strip guides as illustrated, the first actuator strip guides can be a single guide or three or more guides that extend from the actuator strip base and slideably or moveably engage with a single panel guide or three or more panel guides that likewise extend from the panel. Similarly, instead of the second actuator strip guide having inner and outer actuator strip guides, the second actuator strip guide can be a single guide or three or more guides that extend from the actuator strip base and slideably or moveably engage with a single panel guide or three or more panels guide that extend from the panel. In other embodiments, the first actuator strip guide may have multiple actuator strip guides cooperating with multiple corresponding panel guides, while the second actuator strip guide and its cooperating panel guide may have any number of guides different from the first actuator and panel guides, or vice versa. It should likewise be appreciated that instead of the actuator strip guide(s) and panel guide(s) being in the form of a guide post(s) or stick(s), and the panel guide(s) being in the form of rails that cooperatively engage the guide post(s) or stick(s), the panel guides can instead be in the form of the guide post(s) or stick(s) while the panel guides are in the form of the engaging guide rail(s).

Regarding the first, second and third sections 20, 22, 24 of the actuator strip 14, first section 20 includes or defines a substantially uniform width 20a between the first and second actuator guides 26, 28. Second section 22 includes or defines a gradually decreasing width 22a in the direction moving rightwardly from the first section 20a towards the second section 22 as shown in the figures, which is a direction opposite to the direction that the actuator strip is pulled during use, which is illustrated as “PULL DIRECTION” and discussed more below. Third section 24 includes or defines a substantially uniform width 24a that is smaller than the dimeter of the first section 20a. In the illustrated embodiment, the widths 20a, 22a, 24a are each defined by the distance between the inner actuator strip guides 26a, 26b. It should be appreciated, however, that in alternative embodiments, the widths of each of the sections can be defined by the distance between any of the actuator strip guides. For example, the widths of each section can be defined by the distance between an inner actuator guide extending on side of the base to and outer actuator strip guide on the opposite side of the base, by the distance between an inner or outer actuator strip guide on one side of the base to a middle actuator strip guide on an opposite side of the base, or by the distance between a single actuator strip guide on one side of the base to a single actuator strip guide on an opposite side of the base. It should also appreciated that in certain embodiments, neither the width 20a of first section 20 nor the width 24a of third section 24 needs to be substantially uniform so long as the width 24a third section 24 is smaller than the width 20a of section 20 so that the device can provide the wound closing feature described herein.

Referring to FIG. 5, FIG. 5 illustrates an enlarged view of the second section 22 showing the gradually decreasing width 22a in second section 22 along with portions of first and third sections 20, 24 and their respective generally uniform width sections. In particular and viewing from right to left in FIG. 5, width 22a generally decreases along section 22 from the generally larger width 20a of the first section 20 to the generally smaller width 24a of the third section 24, which is shown by the decreasing widths 22a1, to 22a2, to 22a3 to 22a4. The ratio of a length l of section 22 (i.e., the distance along which the width of section 22 gradually decreases) to a difference between the widths 20a and 24a of the first and third sections 20a, 24a, respectively, can be in the range of 8:1 and 12.5:1, and in one particular embodiment in the range of 12.5:1. For example, the length l along which the width of section 22 decreases is 25 mm, while the widths 20a, 24a, of first and third sections 20, 24, respectively, are 5.6 mm and 3.6 mm. Thus, the ratio of the length l along which the width of section 22 decreases to the difference between the widths of the first and third sections 20a, 24a is 12.5:1.

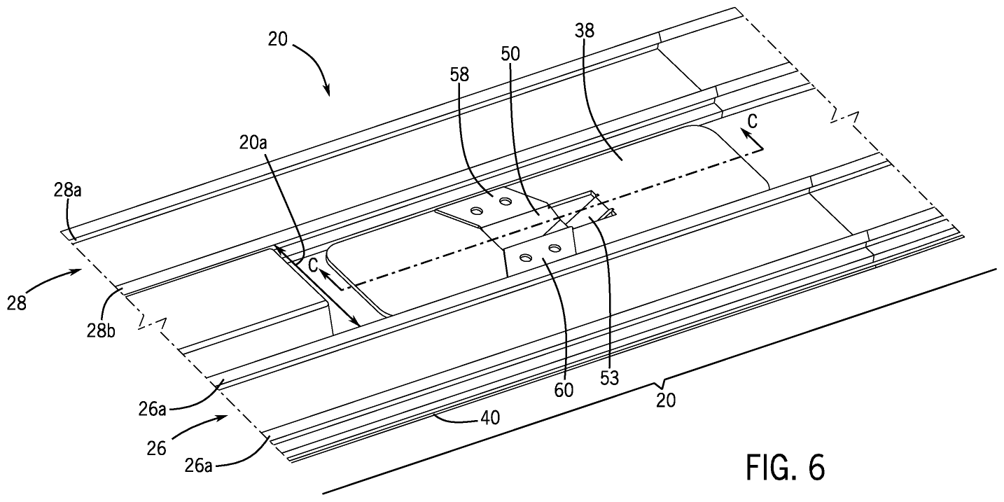

Referring more specifically to the cutter or cutting device 38, FIGS. 6 to 9 illustrate one embodiment of cutting device 38 of the present disclosure. Cutting device 38 is attached to, integrated with or coupled to the first section of actuator strip 14 between the actuator guides 26, 28 and is located in a channel defined between the actuator strip guides of actuator strip 14. As illustrated, cutting device 14 includes a floor 46, a right wall 48, and a left wall 50. Right wall 48 is integrated or attached to the floor 46, and extends upwardly from one side of floor 46 to the actuator strip 14. Left wall 50 is similar integrated or attached to floor 46, and extends upwardly from an opposite side of floor 46 to actuator strip 14. Each of right wall 48, left wall 50 and floor 46 includes a cutting or razor edge 48a, 50a, and 46a, respectively, for cutting the tissue at the donor site. Cutting device 38 further includes wings 60, 58 extending from the right and left walls 50, 48, respectively. Wings 60 and 58 are coupled to a base plate 52, which is coupled to the actuator strip 14. Floor 46 is coupled to a tissue guide 53, which is coupled to or extends from base plate 52, and acts to guide the tissue as the device 10 is cutting the tissue from the donor site in use. It should be appreciated that certain embodiments need not include the separate tissue guide 53. For example, the floor 46 of the cutting device 38 can instead extend all the way to the base plate 52 or to the actuator strip 14.

The floor 46 of cutting device 38 extends from the base 40 of actuator strip 14 at an angle Θ relative to the base 40, such that when the actuator device 14 is pulled to cut or harvest tissue from the donor site (e.g., skin tissue), the cutting device 38 cuts a strip of skin without bunching, resulting in a relatively thin strip of tissue that can be used advantageously, for example, as a coiled-up skin graft construct that includes both a layer of epidermis and dermis (i.e., full thickness), or just the epidermis or the epidermis and an upper portion of the dermis (i.e., partial thickness). Coiling-up a strip of full thickness tissue and placing the coiled tissue in or on a graft site ensures that the dermis and epidermis orientation is consistent across the graft site, which results in better and quicker healing, as discussed in more detail below. In certain embodiments, the width of the thin strip of tissue harvested is, in the range of 0.5 mm (0.019685 inches) to 4.7625 mm ( 3/16 inch) or 1.5875 mm ( 1/16 inch) to 4.7625 mm ( 3/16 inch) for full thickness, and in the range of 12.7 mm (½ inch) to 50.8 mm (2 inch) for partial thickness. In certain embodiments, the angle Θ is in the range of 15 degrees to 60 degrees. The size and dimensions of cutting device 38 can be selected to allow the device 10 to advantageously cut or harvest a strip of tissue suitable for a particular use. For example, the width of the cutting edge 46a of floor 46 (i.e., the distance between walls 48 and 50) can be in the range of 0.15875 mm ( 1/16 inch) to 0.635 mm ( 3/16 inch), and in one particular embodiment can be 1.9 mm wide. The depth at which the edge 46a of floor 46 extends from the underside of the actuator strip base 40 can be in the range of 0.635 mm ( 1/40 inch) to 2.54 mm ( 1/10 inch), and in one particular embodiment can be 2.2 mm. In some embodiments, the cutting device 38 does not need wings and/or a guide plate, and can instead have a single piece extending downwardly from the actuator strip base or the base plate of cutting device 38.

In certain embodiments, the actuator strip 14 is structured so that at least a portion of the actuator strip 14 (including the portion having the cutting device 38) can be removed from the rest of the actuator strip 14 once the device has been pulled to cut the thin strip of tissue. The removal of the portion of the actuator strip allows the device to remain in place over the open wound immediately after pulling the strip to allow the wound to heal, as discussed below. Cutting device 38 itself can also be structured and arranged to be removably coupled to actuator strip 14. For example, in one embodiment, wings 58 and 60 are removably coupled to the base plate 52 via one or more fastener mechanisms such as simple screws or nails. Alternatively or additionally, base plate 52 or the single piece cutting device 38 can be sized and dimensioned to be removably secured between the actuator strip guides (or the channel defined in the base of the actuator strip) with a frictional or snap-fit engagement.

To harvest or cut a thin strip of tissue using the device 10, the device 10 is initially removed from a closed, sterile packaging. Once removed from the sterile packaging, a hermetic seal 44 is secured to the underside of actuator strip 12 and the adhesive backing on the underside of panel 12 are removed so that the panel 12 (and the actuator strip 14 coupled to or integrated with the panel) is secured to the donor site via adhering the panel 12 to the donor site. Once the device 10 is adhered to the donor site, the actuator strip 14 pull tab 36 is pulled in a direction generally leftward as seen in the figures (labeled as “PULLING DIRECTION”) until the stop 34 of the actuator strip 14 engages an edge 12c of the panel 12. The pulling causes actuator strip guides 26, 28 to slide or move in the corresponding panel guides 41, 42, causing the cutter 38 to cut a thin strip of tissue and create an open wound at the donor site. As the actuator strip guides 26, 28 move along the panel guides 41, 42, the gradually decreasing width 22a between the actuator guides 26, 28 causes the width between the panel guides 41, 42 to likewise gradually decrease, which in turn causes the panel 12 (e.g., each of panel pieces 12a, 12b adhered to the donor site) to pull on the tissue on opposite sides of the open wound to at least partially close the open wound. The pulling of the actuator strip 14 also causes the gel pack 32 to apply its antiseptic and anesthetic/analgesic to the donor site, and the bandage 30 to be applied to the open wound. Downward pressure can be applied to the device 10 while pulling, as needed. The actuator strip 14 is pulled to the point where the stop 34 engages the edge 12c of the panel 12, a leftward portion of the actuator strip including the cutting device 38 coupled thereto can be removed from the rest of the actuator strip 14 (e.g., via cutting the portion of the actuator strip) so that the device can remain on the donor site with the bandage in place for a period of time necessary for the wound to heal (e.g., approximately seven days). FIG. 3 illustrates the stop 34 of the actuator strip 14 engaged with the edge portion 12c of the panel 12 to prevent further pulling of the actuator strip 14 and the portion of the actuator strip, which includes the cutting device 38, having been removed from the rest of the actuator strip 14 (e.g. via cutting or tearing the portion of the actuator strip). After removing the portion of the actuator strip, device 10 is then left in place for the wound to heal. The device 10 accordingly functions to harvest a thin strip of tissue in a precisely controlled, single step process that includes opening and closing the wound substantially simultaneously, while applying a bandage and maintaining the sterility of the donor site.

It should accordingly be appreciated that one embodiment of a tissue harvesting and wound closing method of the present disclosure includes securing a panel to a donor site (e.g., adhering the panel), and pulling first and second actuator strip guides along first and second panel guides of the panel, respectively, to cut a strip of tissue from the donor site thereby creating an open wound, and pull together tissue on opposite sides of the open wound to at least partially close the open wound. The method further includes pulling the actuator strip until a stop of the actuator strip engages an edge of the panel, thereby preventing further pulling of the first and second actuator guide strips along the first and second guide panels, and removing (e.g., cutting or tearing) a portion of the actuator strip after said pulling the first and second actuator strips, thereby allowing the device to remain secured to the donor site so that the wound can heal.

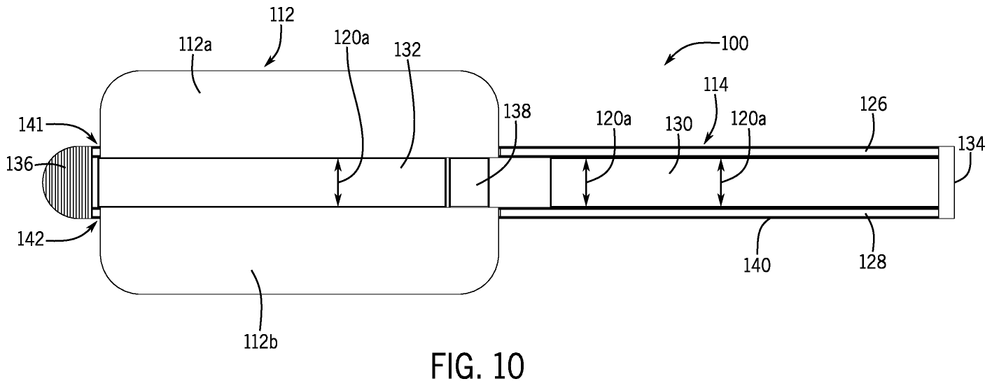

Turning now to FIG. 10, FIG. 10 illustrates one embodiment of a tissue harvesting device 100 of the present disclosure. Device 100 includes many of the same elements described above in connection with device 10. Those elements are marked with the same or similar element numbers. The description of those elements and the function of same, including each of the alternatives discussed above, apply in many respects to like element numbers in FIG. 10. Tissue harvesting device 100 differs from device 10 primarily in that the actuator strip 14 of device 100 does not have a section in which the width between the actuator guides 26, 28 gradually decreases. The width 120a between the actuator guides 126, 128 in device 100 instead remains substantially uniform along the entire length of actuator strip 112. Thus, tissue harvesting device 100 does not provide the substantially simultaneous wound closing function of device 10.

Device 100 can be utilized for cutting a thin strip of full thickness tissue (epidermis and full dermis) or for cutting a thin strip of partial thickness tissue (e.g., epidermis only or epidermis and upper portion of dermis). For harvesting a thin strip of partial thickness tissue, the device 100 can have a uniform width between the actuator guides 126, 128 that is in the range of 12.7 mm (i.e., ½ inch) to 50.8 mm (i.e., 2 inch), and in one particular embodiment is 2.54 mm (i.e., 1 inch). For a full thickness tissue application, the width of the thin strip of tissue harvested can be, for example, in the range of 0.5 mm (0.019685 inch) to 4.7625 mm ( 3/16 inch) or 1.5875 mm (i.e., 1/16 inch) to 4.7625 mm (i.e., 3/16 inch). The angle Θ at which the cutting edge or blade extends from the actuator strip 114 can be in the range of 15 to 60 degrees, and in one particular embodiment, 35 degrees.

It should be appreciated from the foregoing that one embodiment of a tissue harvesting method of the present disclosure includes securing or adhering a panel to a donor site, and pulling first and second actuator strip guides of an actuator strip along first and second panel guides of the panel, respectively, to (i) cut a strip of tissue from the donor site thereby creating an open wound, (ii) apply an anti-septic and anesthetic/analgesic to the donor site, and (iii) place a bandage over the open wound.

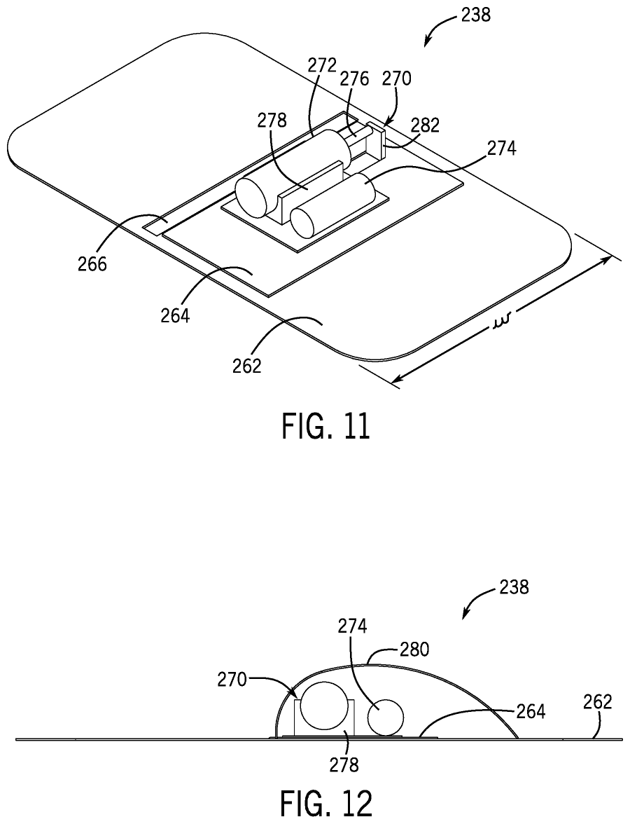

Turning to FIG. 11 and FIG. 12, FIGS. 11 and 12 illustrate one embodiment of a cutting device 238 for coupling or integrating with the actuator strips 14 or 114 of tissue harvesting and wound closing device 10 or tissue harvesting device 100, respectively. Cutting device 238 includes many of the same elements described above in connection with cutting device 38. Those elements are marked with the same or similar element numbers. The description of those elements and the function of same, including each of the alternatives discussed above, apply in many respects to like element numbers in FIGS. 11 and 12. In one particular implementation, cutting device 238 is configured to for use with harvesting a thin strip of partial tissue (e.g., for use with device 100).

Cutting device 238 includes a base 262 and a blade plate 264 coupled to or integrated with base 262. Blade plate 264 can be coupled to the actuator strip 14 similar to the couplings described above with respect to devices 10 and 100. Blade plate 264 includes a sharp or razor portion or edge 266 that extends downwardly through an opening defined in the base 262. Blade plate 264 extends at an angle relative to the actuator strip 14 that is sufficient to cut a thin strip of tissue from a donor site as described above in connection with devices 10 and 100. Like the cutting device 38 discussed above, razor portion 266 of cutting device 238 can also include a right wall, left wall, and middle floor, each of which has a razor edge. Cutting device 238 further includes a blade actuator, power and switch assembly 270. Assembly 270 includes a motor 272, a power source 274 and a switching mechanism. A tissue shield 280 can extend over the assembly 270 to protect the assembly 270 from unwanted and potentially damaging materials. Shield 280 also protects the tissue and donor site from any possible contamination from assembly 270. Motor 272 rests or is coupled to a curved bed 278, and is operatively coupled to a blade plate 264 via a motor output shaft 276 and extending wall 282. When the assembly 270 is switched on, the blade plate 264 is configured to oscillate back-and-forth in a direction that is generally along the width w of the cutting device 238 and base 262. The back-and-forth oscillation causes the razor portion or extending cutting blade to also oscillate back-and-forth, which during the cutting process, reduces the chance that the thin strip of tissue being cut will bunch, particularly when the tissue is a partial skin, which is more elastic and susceptible to such bunching. In one embodiment, the motor and assembly oscillates at a rate of 30 to 60 cycles per second. Partial thickness tissue is typically fenestrated before grafting, and thus to simplify the process, an additional component can be added to fenestrate the partial thickness tissue as it is harvested or collected.

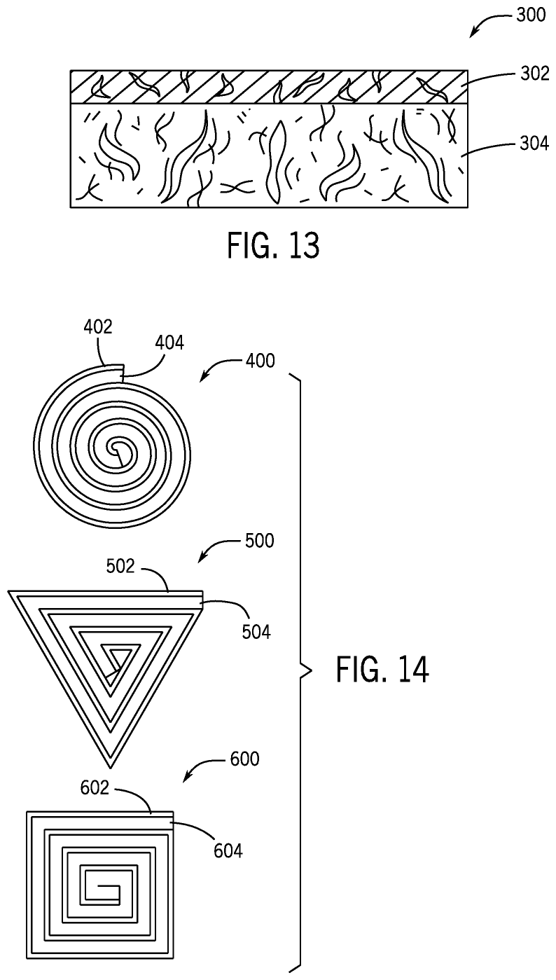

As discussed above, the devices and methods of the present disclosure can be used to cut or harvest a strip of either partial or full thickness tissue. FIG. 13 illustrates one example of a full thickness strip of tissue 300 that can be harvested according to any of the methods and devices described herein, or using any other suitable devices or techniques. The full thickness strip of tissue 300 includes an epidermis layer 302 and a dermis layer 304. In one aspect of the present disclosure, the full thickness tissue strip such as tissue 300 can be advantageously coiled-up for a tissue or skin grafting procedure. For example, the strip of full thickness tissue 300 can be coiled-up into a circular arrangement 400, a triangular arrangement 500 or a rectangular arrangement 600 as illustrated in FIG. 14, each of which includes a respective epidermis layer (402, 502, 602) and dermis layer (404, 504, 604). Once coiled, tissue arrangements 400, 500 or 600 can be placed on or in a graft site for a grafting procedure. The coiled-up arrangements ensure that the orientation of the epidermis and dermis layers is maintained across the graft site, which helps to preserve the dermal architecture, providing better and quicker healing. The coiled-up arrangements are not limited to the arrangements illustrated in FIG. 14, and can be any suitable coiled arrangement that maintains the orientation of the epidermis and dermis layers across the graft site for improved healing.

It should therefore be appreciated that one non-limiting embodiment of the present disclosure includes a skin grafting method in which strip of full thickness tissue is coiled and placed on or in a graft site, and another non-limiting embodiment of the present disclosure includes a coiled-up full thickness strip of harvested tissue for use in a grafting procedure. The arrangement of the coiled-up tissue in either embodiment can be any suitable shape that maintains the orientation of the full-thickness tissue across a graft site, including, but not limited to, circular, triangular, or rectangular arrangements.

While the present disclosure has been described above in connection with particular embodiments and examples, the disclosure is not necessarily so limited, and that numerous other embodiments, examples, uses, modifications and departures from the embodiments, examples and uses are intended to be encompassed by the claims attached hereto.

What is claimed is:

1. A tissue harvesting and wound closing device comprising:
a panel having a length and including:
a first panel guide extending along at least a portion of the length of the panel, and
a second panel guide extending along at least a portion of the length of the panel; and
an actuator strip coupled to the panel and including:
a base having a length and defining first, second, and third sections,
a first actuator strip guide extending along at least a portion of the length of the base and slideable along the first panel guide,
a second actuator strip guide extending along said at least a portion of the length of the base and slideable along the second panel guide,
wherein (a) the first section of the base has a substantially uniform width between the first actuator strip guide and the second actuator strip guide, (b) the second section of the base has a gradually decreasing width between the first actuator strip guide and the second actuator strip guide, and (c) the third second section of the base has a substantially uniform width between the first actuator strip guide and the second actuator strip guide that is smaller than the substantially uniform width of the first section, and
a cutter coupled to the first section of the base and extending at an angle relative to the base, and
wherein when the device is secured to a donor site and the actuator strip is pulled, the first and second actuator strip guides slide along the first and second panel guides, respectively, such that (i) the cutter cuts a strip of tissue from the donor site thereby creating an open wound, and (ii) the panel pulls together proximal tissue on opposite sides of the open wound to at least partially close the open wound.

2. The tissue harvesting and wound closing device of claim 1, wherein: (i) the first panel guide includes an inner panel guide and an outer panel guide, the second panel guide includes an inner panel guide and an outer panel guide, the first actuator strip guide includes an inner actuator strip guide and an outer actuator strip guide, the second actuator strip guide includes an inner actuator strip guide and an outer actuator strip guide, the inner and outer actuator strip guides of the first actuator strip guide slideable along the inner and outer panel guides of the first panel guide, respectively, and the inner and outer actuator strip guides of the second actuator strip guide slideable along the inner and outer panel guides of the second panel guide, (ii) the first section has a substantially uniform width between the inner actuator strip guide of the first actuator strip guide and the inner actuator strip guide of the second actuator strip guide, (iii) the second section has a gradually decreasing width between the inner actuator strip guide of the first actuator strip guide and the inner actuator strip guide of the second actuator strip guide, and (iv) the third second section has a substantially uniform width between the inner actuator strip guide of the first actuator strip guide and the inner actuator strip guide of the second actuator strip guide, the substantially uniform width of the third section smaller than the substantially uniform width of the first section.

3. The tissue harvesting and wound closing device of claim 1, wherein a channel is defined between the first and second actuator strip guides, the cutter positioned in the channel.

4. The tissue harvesting and wound closing device of claim 1, wherein the panel is removably coupled to the actuator strip.

5. The tissue harvesting and wound closing device of claim 1, wherein the angle of the cutter relative to the base of the actuator strip is between 15° and 60°.

6. The tissue harvesting and wound closing device of claim 1, wherein the panel includes (i) a first panel piece, the first panel piece including the first panel guide, and (ii) a second panel piece, the second panel piece including the second panel guide.

7. The tissue harvesting and wound closing device of claim 1, which includes (i) an anti-microbial bandage and an antiseptic and anesthetic gel pack positioned in the third and first sections of the actuator strips, respectively, such that when the panel is secured to the donor site and the actuator strip is pulled, antiseptic and anesthetic are applied to the donor site from the gel pack and the bandage is placed over the at least partially closed wound.

8. The tissue harvesting and wound closing device of claim 1, wherein the actuator strip includes a stop extending from the actuator strip, wherein said pulling of the actuator strip is prevented when the stop engages an edge of the panel.

9. The tissue harvesting and wound closing device of claim 1, wherein the first panel guide includes clamping rails slideably coupled to the first actuator strip guide, and the second panel guide includes clamping rails slideably coupled to the second actuator strip guide.

10. The tissue harvesting and wound closing device of claim 1, wherein the actuator strip includes a single piece having a semi-rigid material.

11. The tissue harvesting and wound closing device of claim 1, wherein the cutter includes a width having a range of 0.5 mm to 4.7625 mm that allows the device to cut a strip of full thickness tissue.

12. The tissue harvesting and wound closing device of claim 1, wherein the ratio of a length of the second section to the difference between the widths of the first and third sections, respectively, is in the range of 8:1 and 12.5:1.

13. The tissue harvesting and wound closing device of claim 1, wherein the actuator strip defines an opening, the cutter coupled to the actuator strip at or near an edge of the opening.

14. The tissue harvesting and wound closing device of claim 1, wherein the cutter includes (i) a middle floor positioned at an angle relative to the base that is sufficient to cut the tissue from the donor site, (ii) a right wall attached to and extending upwardly from the middle floor, and (ii) a left wall attached to and extending upwardly from the middle floor, each of the middle floor, the left wall, and the right wall including a blade edge structured and arranged to cut the tissue when the actuator is pulled.

15. The tissue harvesting and wound closing device of claim 1, wherein (a) the panel includes an adhesive for securing the device to the donor site, and (b) when the panel is adhered to the donor site and the actuator strip is pulled, the first and second actuator guides slide along the first and second panel guides, respectively, such that (i) the cutter cuts the strip of tissue from the donor site creating the open wound, and (ii) the panel pulls together proximal tissue on opposite sides of the open wound to at least partially close the open wound.

16. The tissue harvesting and wound closing device of claim 1, wherein the cutter is removably coupled to the actuator strip.

17. A tissue harvesting and wound closure method comprising:
securing a panel to a donor site; and
pulling first and second actuator strip guides along first and second panel guides of the panel, respectively, to (i) cut a strip of tissue from the donor site thereby creating an open wound, and (ii) pull together proximal tissue on opposite sides of the open wound to at least partially close the open wound.

18. The tissue harvesting and wound closing method of claim 17, wherein said securing the panel to the donor site includes adhering the panel to the donor site.

19. The tissue harvesting and wound closing method of claim 17, wherein said pulling of the first and second actuator guide strips along the first and second panel guides includes pulling the actuator strip until a stop of the actuator strip engages an edge of the panel, thereby preventing further pulling of the first and second actuator guide strips along the first and second guide panels.

20. The tissue harvesting and wound closing method of claim 17, wherein the strip of tissue includes full thickness tissue.

21. The tissue harvesting and wound closing method of claim 17, which includes removing a portion of the actuator strip after said pulling the first and second actuator strips, and allowing the remaining portion of the actuator strip and the panel to be secured to the donor site so that the wound can heal.

22. The tissue harvesting and wound closing method of claim 21, wherein said removing a portion of the actuator strip includes cutting a portion of the actuator strip.

23. A tissue harvesting and wound closing actuator strip comprising:
a base having a length and defining first, second and third sections;
a first actuator strip guide extending along at least a portion of the length of the base and structured to be slideable along a first panel guide of a panel;
a second actuator strip guide extending along said at least the portion of the length of the base and structured to be slideable along a second panel guide of a second panel,
wherein (i) the first section of the base has a substantially uniform width between the first actuator strip guide and the second actuator strip guide, (ii) the second section of the base has a gradually decreasing width between the first actuator strip guide and the second actuator strip guide, and (iii) the third second section of the base has a substantially uniform width between the first actuator strip guide and the second actuator strip guide that is smaller than the substantially uniform width of the first section, and
a cutter positioned in the first section of the base at an angle sufficient to cut a strip of tissue from a donor site, and
wherein when the actuator strip is secured to the donor site and the actuator strip is pulled, the first and second actuator guides slide along the first and second panel guides, respectively, such that (i) the cutter cuts a strip of the tissue from the donor site thereby creating an open wound, and (ii) the panel pulls together proximal tissue on opposite sides of the open wound to at least partially close the open wound.

24. A tissue harvesting device comprising:
a panel having a length and including:
a first panel guide extending along at least a portion of the length of the panel, and
a second panel guide extending along at least a portion of the length of the panel; and
an actuator strip coupled to the panel and including:
a base having a length,
a first actuator strip guide extending along at least a portion of the length of the base and slideable along the first panel guide,
a second actuator strip guide extending along said at least a portion of the length of the base and slideable along the second panel guide,
a bandage positioned on the base,
an antiseptic and anesthetic/analgesic gel pack positioned on the base, and
a cutter extending at an angle relative to the base sufficient to cut a strip of tissue from a donor site, and
wherein when the device is secured to the donor site and the actuator strip is pulled, the first and second actuator strip guides slide along the first and second panel guides, respectively, such that (i) the cutter cuts the strip of tissue from the donor site thereby creating an open wound, (ii) the antiseptic and anesthetic/analgesic from the gel pack is applied to the donor site, and (iii) the bandage is placed over the open wound.

25. A tissue harvesting method comprising:
securing a panel to a donor site; and
pulling first and second actuator strip guides of an actuator strip along first and second panel guides of the panel, respectively, to (i) cut a strip of tissue from the donor site thereby creating an open wound, (ii) apply an anti-septic and anesthetic/analgesic to the donor site, and (iii) place a bandage over the open wound.

26. The tissue harvesting and wound closing method of claim 25, wherein the strip of tissue includes (i) full thickness tissue, or (ii) partial thickness tissue.

27. The tissue harvesting and wound closing method of claim 25, which includes removing a portion of the actuator strip after said pulling the first and second actuator strips, thereby allowing the device to remain secured to the donor site so that the wound can heal.

28. The tissue harvesting and wound closing method of claim 25, where said removing at least a portion of the actuator strip after said pulling the first and second actuator strips includes cutting said portion of the actuator strip.

29. A skin graft method comprising:
coiling a strip of full thickness tissue; and
placing the coiled full thickness tissue on a graft site.

30. The skin grafting method of claim 29, wherein said coiling includes coiling the full-thickness tissue into a shape that maintains the orientation of the full-thickness tissue.

31. The skin grafting method of claim 30, wherein the shape is one of circular, triangular, or rectangular.
